(19) United States
(12) Patent Application Publication
(10) Pub. No.: US 2026/0207079 A1
(43) Pub. Date: Jul. 23, 2026

(54) WALKING MAT AND METHOD FOR JUDGING WALKING CONDITION USING WALKING MAT

(71) Applicant: IUCF-HYU (INDUSTRY-UNIVERSITY COOPERATION FOUNDATION HANYANG UNIVERSITY), Seoul (KR)

(72) Inventors: Jieun KIM, Seoul (KR); Wooyoung JEONG, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 19/139,074

(22) PCT Filed: Nov. 28, 2023
(86) PCT No.: PCT/KR2023/019386

§ 371 Date: Jun. 13, 2025

(30) Foreign Application Priority Data
Dec. 14, 2022 (KR) 10-2022-0174623

(51) Int. Cl. A61B 5/103; A61B 5/00; A63B 21/00

(52) U.S. Cl. CPC A61B 5/1038; A61B 5/0022; A61B 5/7282; A61B 5/746; A63B 21/4037; A63B 2220/833

(57) ABSTRACT

The present invention relates to a walking mat and a method for judging a walking condition using the same, and more particularly, to a walking mat and a method for judging a walking condition that can determine a user's walking condition. A walking condition judging apparatus according to an embodiment of the present invention includes a modem configured to receive sensing information from a connected walking mat and output the sensing information, a memory in which at least one of predetermined normal walking information and predetermined normal standing information is stored, and a processor. The memory stores program instructions executable by the processor to update the normal walking information or the normal standing information when the sensing information is received, and to determine whether an emergency situation has occurred when the sensing information is not received. The walking mat may generate the sensing information through an internal pressure sensor and transmit the sensing information to the walking condition judging apparatus.

### TECHNICAL FIELD

The present disclosure relates to a walking mat and a method for judging a walking condition using the same, and more particularly, to a walking mat and a method for judging a walking condition that can determine a user's walking condition.

### BACKGROUND ART

Human postural control is achieved through the balance between sensory information and movement. Motor control involves the coordination of postural responses of trunk and lower limb muscles, while sensory processing refers to the organization of information received from somatosensory inputs (including tactile and proprioceptive senses), vision, and the vestibular system. However, these sensory functions gradually deteriorate with age, which leads to frequent fall-related accidents among the elderly. People with impaired sensory function due to accidents are also exposed to a high risk of falls.

To prevent falls among the elderly and infirm, simple exercises to improve balance are recommended. However, exercise alone has limitations in preventing such accidents. Therefore, there is a need for a device capable of immediately providing elderly users with specific information, such as whether they are standing with sufficient balance to walk, or whether there is a risk of falling while walking.

Meanwhile, as society ages, the number of elderly people living alone is increasing. These individuals may suffer serious injuries or even death from minor falls due to a lack of timely assistance. Furthermore, crimes targeting solitary elderly individuals, such as burglaries, are also on the rise. Therefore, there is a need for a system that can automatically detect such accidents and quickly request help.

### DESCRIPTION OF EMBODIMENTS

### Technical Problem

The present disclosure provides a walking mat and a method for judging a walking condition using the same, which can automatically detect accidents involving elderly individuals living alone and promptly request assistance.

### Solution to Problem

According to an embodiment of the present disclosure, a walking condition judging apparatus includes: a modem configured to receive sensing information from a connected walking mat and output the received information; a memory in which one or more of predetermined normal walking information and predetermined normal standing information is stored; and a processor. The memory stores program instructions executable by the processor to update the normal walking information or the normal standing information when the sensing information is received, and to determine whether an emergency situation has occurred when the sensing information is not received. The walking mat generates and transmits the sensing information through pressure sensors formed therein.

In one embodiment, the memory may store program instructions to compare an unrecognized accumulated time with a predetermined threshold time and determine whether an emergency situation has occurred when the sensing information is not received.

In one embodiment, the memory may store program instructions to generate a confirmation signal and transmit the confirmation signal to the walking mat when the accumulated time exceeds the threshold time, and the walking mat may output a user confirmation request when the confirmation signal is received.

In one embodiment, the memory may store program instructions to determine that situation is not an emergency when a response message is received, and the walking mat may transmit the response message to the modem when a user operation is input after outputting the user confirmation request.

In one embodiment, the memory may store program instructions to transmit an emergency message to a pre-registered contact when the response message is not received within a predetermined time after the confirmation signal is transmitted.

In one embodiment, when the sensing information is received and one foot is recognized, the memory may store program instructions to generate at least one of single-foot weight information, stride information, and walking speed information according to a predetermined method using the sensing information, and determine whether the information corresponds to an authorized user based on the generated information.

In one embodiment, the memory may store program instructions to generate first user information using the following Equation 1 and to determine whether the user is an authorized user by comparing the first user information with the normal walking information.

$${U}_{1} = {α}_{1} {P}_{1} + {β}_{1} W + {γ}_{1} V \qquad [ Equation 1 ]$$

Here, U₁ is the first user information, α₁, β₁, and γ₁ are positive real-valued preset coefficients, P₁ is the single-foot weight information, W is the stride information, and V is the walking speed information.

In one embodiment, the memory may store program instructions to output an unauthorized user notification when a difference between the first user information and the normal walking information exceeds a predetermined error range, and, in response to an authorized user notification input based on a user operation, update the average value of the normal walking information using the first user information, and adjust the coefficients α₁, β₁, and γ₁ so that the first user information becomes equal to the updated normal walking information.

In one embodiment, the memory may store program instructions to transmit an emergency message to a pre-registered contact when the authorized user notification is not input.

In one embodiment, when both feet are recognized and one foot is not recognized within a predetermined time, the memory may store program instructions to generate at least one of both-foot weight information, foot size information, and foot width information using the sensing information according to a predetermined method, and to determine whether the user is an authorized user based on the generated information.

In one embodiment, the memory may store program instructions to generate second user information using the following Equation 2 and to determine whether the user is an authorized user by comparing the second user information with the normal standing information.

$${U}_{2} = {α}_{2} {P}_{2} + {β}_{2} S + {γ}_{2} B \qquad [ Equation 2 ]$$

Here, U₂ is the second user information, α₂, β₂, and γ₂ are positive real-valued preset coefficients, P₂ is the both-foot weight information, S is the foot size information, and B is the foot width information.

In one embodiment, the memory may store program instructions to output an unauthorized user notification when a difference between the second user information and the normal standing information exceeds a predetermined error range, and, in response to an authorized user notification input based on a user operation, update the average value of the normal standing information using the second user information, and adjust the coefficients α₂, β₂, and γ₂ so that the second user information becomes equal to the updated normal standing information.

In one embodiment, the memory may store program instructions to transmit an emergency message to a pre-registered contact when the authorized user notification is not input.

### Advantageous Effects of Disclosure

According to the present invention, it is possible to provide a walking mat and a method for judging a walking condition using the same, which can automatically detect accidents involving elderly individuals living alone and promptly request assistance.

### BRIEF DESCRIPTION OF DRAWINGS

To more fully understand the drawings referenced in the detailed description of the present invention, brief descriptions of each drawing are provided below.

FIG. 1 is a diagram illustrating a walking condition judging system using a walking mat according to an embodiment of the present invention.

FIGS. 2 and 3 are diagrams illustrating the physical configuration of the walking mat according to an embodiment of the present invention.

FIG. 4 is a diagram illustrating the logical configuration of a walking condition judging apparatus using a walking mat according to an embodiment of the present invention.

FIG. 5 is a flowchart illustrating a method for judging a walking condition according to an embodiment of the present invention.

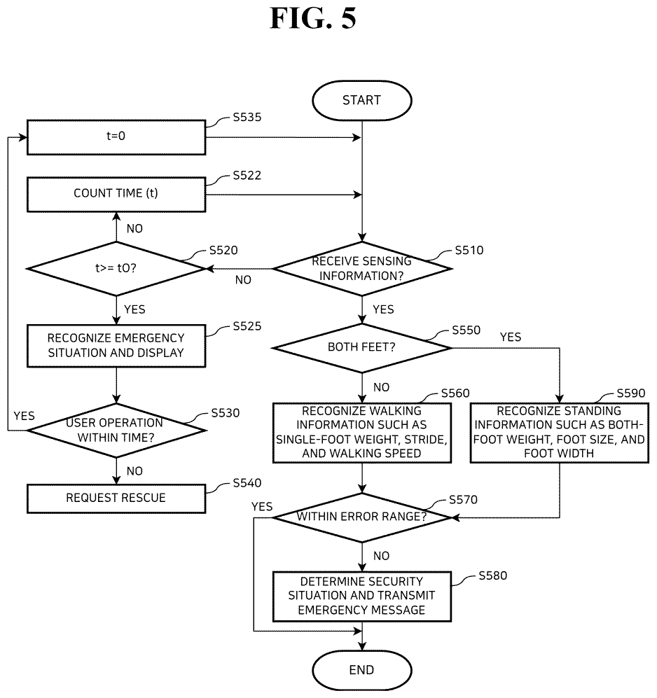

### BEST MODE

The technical concept of the present disclosure may be subject to various modifications and may have various embodiments. Specific embodiments are illustrated in the drawings and described in detail herein. However, this is not intended to limit the technical concept of the present disclosure to specific forms, and it should be understood to include all modifications, equivalents, and alternatives within the scope of the technical concept of the present disclosure.

In describing the technical concept of the present disclosure, detailed descriptions of related known technologies may be omitted if they are deemed to obscure the gist of the present disclosure. In addition, numerical labels (e.g., first, second, etc.) used in the description are merely for distinguishing one component from another.

As used herein, when one component is described as being “connected to” or “coupled to” another component, it should be understood that the component may be directly connected or coupled to the other component, or may be indirectly connected or coupled through another component, unless otherwise stated.

The terms such as “unit,” “device,” or “module” used herein refer to a unit for processing at least one function or operation, and may be implemented in hardware such as a processor (CPU, GPU, APU, DSP, ASIC, FPGA, etc.), software, or a combination of hardware and software.

Additionally, the classification of the components described herein is merely based on their respective main functions. Accordingly, two or more components may be combined into one, or a single component may be subdivided into two or more subcomponents by function. Each component may perform not only its main function but also part or all of the functions performed by other components. Conversely, part of the main function of a component may be dedicated to and performed by another component.

Hereinafter, various embodiments according to the technical concept of the present disclosure will be described in detail.

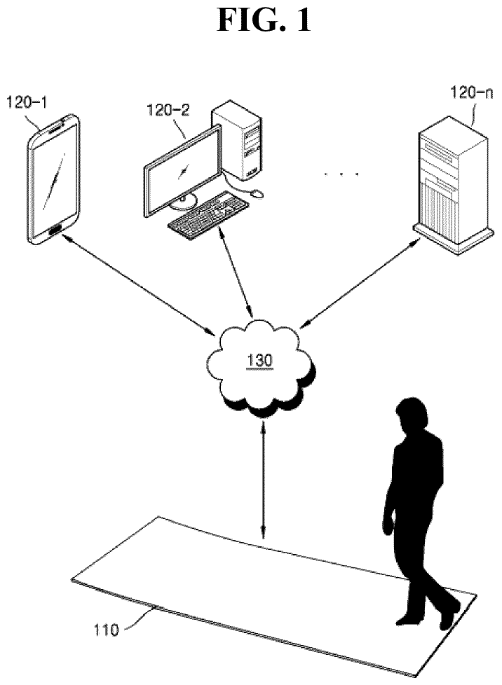

FIG. 1 is a diagram illustrating a walking condition judging system using a walking mat according to an embodiment of the present invention.

Referring to FIG. 1, the walking condition judging system 100 may include one or more walking mats 110 and a walking condition judging device 120 such as a portable terminal 120-1, a personal computer 120-2, or a server 120-n. The walking mat 110 and the walking condition judging device 120 may be connected via a wired or wireless network 130. The network 130 may be any type of network that can connect the walking mat 110 and the walking condition judging device 120. For example, the network 130 may be a short-range communication network such as Bluetooth, Zigbee, or Wi-Fi. In another example, the network 130 may be a long-range communication network such as the Internet or a mobile communication network.

The walking condition judging system 100 may determine whether the person standing on the walking mat 110 is an authorized user. The reason is that an intruder targeting the residence of an elderly person living alone could be standing on the walking mat 110. Therefore, the walking condition judging system 100 may sense the pressure of both feet of the user standing on the walking mat 110 and compare the sensed data with stored information about authorized users to determine whether the person on the mat is legitimate. In addition, if no user stands on the walking mat 110 for a certain period of time, the walking condition judging system 100 may determine whether an emergency situation has occurred.

The operations of the walking condition judging system 100 described above will now be described in more detail.

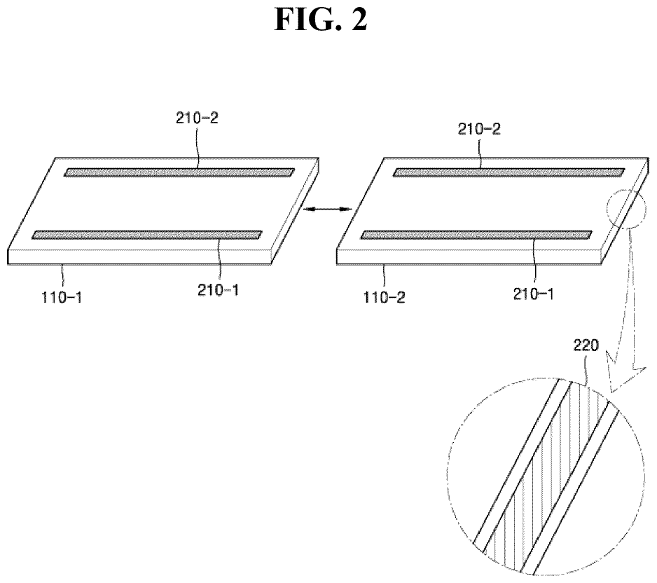

FIGS. 2 and 3 are diagrams illustrating the physical configuration of the walking mat according to an embodiment of the present invention.

First, referring to FIG. 2, the physical configuration of the walking mat 110 according to an embodiment of the present invention will be described.

A plurality of walking mats 110 may be electrically connected to each other. For example, each of a first walking mat 110-1 and a second walking mat 110-2 may be provided with a connection terminal. The connection terminal of the first walking mat 110-1 and the connection terminal of the second walking mat 110-2 may be directly connected, or may be electrically connected by connecting cables or the like.

In addition, light units 210-1 and 210-2 may be formed on both sides of the walking mat 110. For example, a first light unit 210-1 may be formed on the left side of the upper surface of the walking mat 110, and a second light unit 210-2 may be formed on the right side. When the user is standing on the upper surface of the walking mat 110 in a walkable condition, the first and second light units 210-1 and 210-2 may emit light of the same color, thereby visually indicating to the user that walking is possible. In contrast, when the user is standing in a condition in which a risk of falling during walking, the first and second light units may emit light of different colors, thereby visually indicating that walking is not recommended.

Furthermore, when the user is walking normally on the upper surface of the walking mat 110, the first and second light units may emit light of the same color to indicate normal walking. Conversely, if the user is walking abnormally, the light units may emit different colors to visually indicate an abnormal walking condition. If the abnormality originates from the left foot, the first light unit 210-1 may emit a specific color to indicate that the abnormality is due to the left foot. Likewise, if the abnormality is caused by the right foot, the second light unit 210-2 may emit the same color to indicate the cause.

Although FIG. 2 illustrates an example in which the light units are formed on both sides of the walking mat 110, the light units may be formed in any location that is visually observable by the user during standing or walking. Referring to FIG. 3, an example is shown in which a plurality of individual light units (hereinafter collectively referred to as 210) are arranged in a matrix on the top surface of the walking mat 110. In this case, the individual light units 210 may be mapped to pressure sensors 220, and the light units located at pressure-sensing positions may emit light, allowing the user to intuitively align their center of gravity with the illuminated light units.

Referring back to FIG. 2, pressure sensors 220 may be formed in the walking mat 110. For example, the pressure sensors may be embedded inside the walking mat 110. The pressure sensors 220 may be any type of sensor capable of sensing pressure applied to the upper surface of the mat, regardless of shape or type. Accordingly, when the user steps onto the walking mat 110, the sensors may detect pressure from one or both feet.

In addition, a communication modem (not shown) and a processor (not shown) may be included in the walking mat 110. The communication modem of the walking mat 110 may serve to transmit sensing information generated by the pressure sensors 220 to the walking condition judging device 120, or to receive information transmitted from the walking condition judging device 120. The processor of the walking mat 110 may process the information generated by the pressure sensors 220 so that the information is transmitted to the walking condition judging device 120, and may also process information received from the walking condition judging device 120 so that corresponding functions are executed.

FIG. 3 is a diagram illustrating a logical configuration of a walking condition judging apparatus using a walking mat according to an embodiment of the present invention.

Referring to FIG. 3, the walking condition judging device 120 may include a modem 410, a memory 420, a processor 430, and a display 440.

The modem 410 may be a communication device that enables the walking condition judging device 120 to be electrically connected to the walking mat 110. In other words, the modem 410 may allow the walking condition judging device 120 to connect to the network 130.

The memory 420 may be a data storage medium such as a hard disk drive (HDD) or solid state drive (SSD). Program instructions executable by the processor 430 may be stored in the memory 420. In particular, sensing information received from the walking mat 110 may be stored in the memory 420. In addition, the memory 420 may store program instructions for determining whether an authorized user is on the walking mat 110 using the sensing information.

The processor 430 may be a central processing unit (CPU), a graphics processing unit (GPU), or an equivalent computing device that controls overall operations of the walking condition judging device 120. Specifically, the processor 430 may automatically determine whether an authorized user is present on the walking mat 110 by using the sensing information and the program instructions stored in the memory 420.

The display 440 may be a device that visually outputs text, images, or videos input by the processor 430 so that the user can perceive necessary information. The display may include, for example, an LED or LCD screen.

Although not illustrated, the walking condition judging device 120 may further include an input device (not shown) such as a touchpad or keypad, which can generate a signal based on a user operation and output it to the processor 430.

Hereinafter, the operations of the processor 430 will be described in more detail.

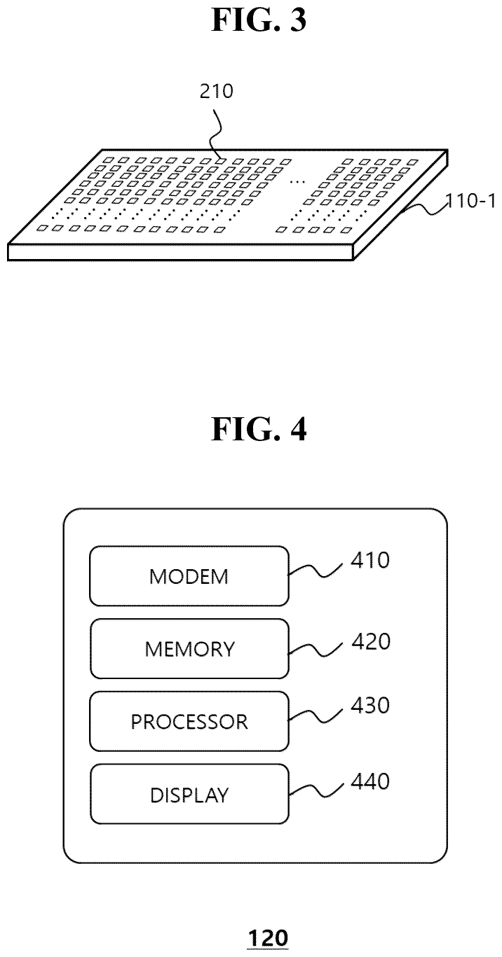

FIG. 4 is a flowchart illustrating a method for judging a walking condition according to an embodiment of the present invention.

Referring to FIG. 4, the method for judging a walking condition according to the present embodiment may be performed by the processor 430 of the walking condition judging device 120 executing program instructions stored in the memory 420. However, for clarity and convenience of explanation, the following description will refer to the processor 430 as the acting entity.

In step S510, the processor 430 may determine whether sensing information has been received. When the user steps onto the walking mat 110, the mat may generate sensing information—i.e., information regarding pressure caused by the user's weight—through internal pressure sensors, and transmit the sensing information to the walking condition judging device 120. Accordingly, the processor 430 may determine whether the modem 410 has received sensing information from the walking mat 110.

In step S520, the processor 430 may determine whether a predetermined threshold time (t0) has elapsed without receiving any sensing information from the walking mat 110. If the walking mat 110 remains powered on but unused for an extended period, the processor 430 may determine that the user may be in an emergency situation. Therefore, the processor 430 may compare the accumulated time counted from the moment the last sensing information was received with the predetermined threshold time.

In step S522, if the accumulated time is less than the threshold time, the processor 430 may continue counting the accumulated time.

In step S525, if the accumulated time exceeds the threshold time, the processor 430 may recognize that an emergency has occurred and generate a confirmation signal to reconfirm whether an emergency situation exists. The processor 430 may then transmit the generated confirmation signal to the walking mat 110. The processor 430 may also control the confirmation signal to be output via the display 440. When the walking mat 110 receives the confirmation signal, the walking mat 110 may activate the light units 210 to prompt the user for confirmation. Hereinafter, the illumination of the light units 210 in response to the confirmation signal will be referred to as a “user confirmation request.” The user may visually perceive the confirmation request via the display 440 and/or the light units 210 of the walking mat.

In step S530, the processor 430 may check whether a user operation is input within a predetermined time after the confirmation signal is generated. If the user properly perceives the confirmation signal via the display 440 and/or the light units 210 of the walking mat, the situation may not be an emergency. In this case, the user may step onto the walking mat 110 or operate a separately provided input device (not shown), whereby the walking mat 110 generates a response message and transmits it to the modem 410. The modem 410 may then output the response message to the processor 430, enabling the processor to recognize that the situation is not an emergency. Alternatively, the user may operate the input device (not shown) of the walking condition judging device 120 after recognizing the displayed confirmation signal to generate the response message, by which the processor 430 may also recognize that the situation is not an emergency.

In step S535, when the processor 430 recognizes from the response message that the situation is not an emergency, the processor may reset the accumulated time to zero.

In step S540, if the response message is not received within a predetermined time after the confirmation signal is transmitted, the processor 430 may transmit an emergency message to a pre-registered contact to notify that an emergency has occurred to the user. The contact information may be pre-stored in the memory 420. For example, the processor 430 may send a text message (SMS) containing a phrase such as “An emergency has occurred to the user” to the guardian's mobile phone.

In step S550, when sensing information is received from the walking mat 110, the processor 430 may analyze the received sensing information to determine whether the information pertains to both feet of the user. The purpose of analyzing the received sensing information is to determine whether the user is standing upright on both feet or in the process of walking.

In step S560, when multiple pieces of sensing information are sequentially input from the walking mat 110 and correspond to one foot, the processor 430 may recognize walking information based on the input. That is, if only one pressure-detected position is included in each piece of sensing information, the processor 430 may determine that the user is currently walking. For example, assume that first to third sensing data are received. Each piece of sensing information may include the following:
(1) The unique identifier of the walking mat 110 (when multiple walking mats are electrically connected)
(2) The position where pressure is detected (2D coordinates)
(3) The pressure intensity (i.e., the intensity of pressure per coordinate in item (2))

Accordingly, the processor 430 may use the first position and pressure from the first sensing information, the second position and pressure from the second sensing information, and the third position and pressure from the third sensing information to generate information such as the user's stride, walking speed, and body weight. For example, the processor 430 may extract the center point (e.g., center of mass of detected coordinates) of each of the first, second, and third positions to generate stride information (hereinafter referred to as “stride information”). The stride information may correspond to the distance between the first and third positions.

In another example, the processor 430 may use the first, second, and third pressure values to generate information related to the pressure applied to the ground during walking (e.g., the average pressure value at the first position), hereinafter referred to as “single-foot weight information.”

In yet another example, the processor 430 may use the reception times of each of the first to third sensing information and the movement distances between their respective center points to generate information about the user's walking speed (hereinafter referred to as “walking speed information”).

The processor 430 may generate first user information using the stride information, single-foot weight information, and walking speed information based on a predetermined Equation 1.

$${U}_{1} = {α}_{1} {P}_{1} + {β}_{1} W + {γ}_{1} V \qquad [ Equation 1 ]$$

Here, U₁ denotes the first user information, and α₁, β₁, and γ₁ are predetermined positive real-valued coefficients. P₁ denotes the single-foot weight information, W denotes the stride information, and V denotes the walking speed information.

The user's stride may change over time, body weight may increase or decrease, and walking speed may not remain constant. Therefore, the processor 430 may normalize the walking-related data and generate it as a single value representing the first user information.

In addition, the processor 430 may compare the first user information with predetermined normal walking information to determine whether the sensing information corresponds to an authorized user. For example, the processor 430 may determine that the sensing information is from an unauthorized user if the difference between the first user information and the normal walking information exceeds a preset error range. The normal walking information may be pre-stored in the memory 420. The normal walking information may be automatically set during the initial setup of the walking mat 110 based on the user's single-foot weight, walking speed, and/or stride. In this case, the initial values of α₁, β₁, and γ₁ may be preset default values. As will be described later, α₁, β₁, and γ₁ may be subsequently updated based on the first user information generated over time.

Although the above example describes generating the first user information using three pieces of sensing data, more sensing data may be used to generate the first user information. In that case, the values P and W used in the first user information may be the averages of the respective values extracted from each piece of sensing data. In addition, when sensing data collection for one foot ends, the data received up to that point may be used to generate the first user information. Using the data received up to that point enables the system to accommodate users, such as elderly individuals, who may walk intermittently with frequent pauses.

In step S570, if the difference between the first user information and the normal walking information exceeds a predetermined error range, the processor 430 may output an unauthorized user notification. For example, the error range may be set to 5% of the normal walking information. In this case, if the difference between the first user information and the normal walking information is within 5% of the stored normal walking information, the processor 430 may determine that the first user information corresponds to an authorized user. On the other hand, if the difference exceeds the 5% range, the processor 430 may output an unauthorized user notification.

The processor 430 may transmit the generated unauthorized user notification to the walking mat 110. The processor 430 may also control the unauthorized user notification to be output via the display 440. When the walking mat 110 receives the unauthorized user notification, the walking mat 110 may activate the light units 210 to prompt the user for confirmation. The user may visually confirm the unauthorized user notification via the display 440 and/or the light units 210 of the walking mat.

After generating the unauthorized user notification, the processor 430 may check whether a user operation is input within a predetermined time. The authorized user may recognize the unauthorized user notification via the display 440 and/or the light units 210 of the walking mat and respond to confirm their presence. For this purpose, the user may operate a separately provided input device (not shown), and the walking mat 110 may generate an authorized user notification and transmit it to the modem 410. The modem 410 may then output the authorized user notification to the processor 430. In this way, the processor 430 may recognize that the first user information corresponds to an authorized user. Alternatively, the user may recognize the displayed unauthorized user notification and operate the input device (not shown) of the walking condition judging device 120 to generate the authorized user notification, whereby the processor 430 may also recognize the user as authorized.

In addition, when the authorized user notification is received, the processor 430 may update the average value of the normal walking information using the first user information that exceeds the error range. The processor 430 may also adjust the coefficients α₁, β₁, and γ₁ so that the first user information becomes equal to the updated normal walking information. For example, the updated normal walking information may be adjusted by half the difference from the first user information. Accordingly, the processor 430 may change α₁, β₁, and γ₁ so that the first user information matches the updated normal walking information. Multiple combinations of α₁, β₁, and γ₁ can result in the first user information becoming equal to the updated normal walking information. In such cases, the processor 430 may select the combination in which the value of α₁ is the largest.

In step S580, if the authorized user notification is not received within a predetermined time after the unauthorized user notification is transmitted, the processor 430 may determine that the user is in a critical security situation and transmit an emergency message to a pre-registered contact. As a result, the system may notify a guardian that an emergency has occurred. The contact information for the guardian may be stored in advance in the memory 420, and the processor 430 may send an emergency message (e.g., SMS) containing a phrase such as “An emergency has occurred to the user” to the guardian's mobile phone.

In step S590, based on the analysis of the sensing information received from the walking mat 110 in step S550, if the processor 430 determines that the user is standing upright on both feet, the processor may recognize standing information using the sensing data. Specifically, if the sensing information includes two pressure-detected positions, the processor 430 may determine that the user is in a standing state. In such a case, only one piece of sensing information may be received, without sequential input, after a predetermined time has elapsed. Accordingly, the processor 430 may use the two pressure-detected positions included in the sensing information to generate standing information including the user's body weight, foot size, and foot width.

For example, the processor 430 may recognize the average pressure value of the coordinates included in the sensing information as the both-foot weight information. Additionally, the processor 430 may recognize the user's foot size information (hereinafter referred to as “foot size information”) by analyzing the area and/or length over which pressure is detected in the sensing data. The processor 430 may also recognize the user's foot width information (hereinafter referred to as “foot width information”) based on the area and/or width where pressure is detected in the sensing data.

In addition, the processor 430 may generate second user information using the following Equation 2.

$${U}_{2} = {α}_{2} {P}_{2} + {β}_{2} S + {γ}_{2} B \qquad [ Equation 2 ]$$

Here, U₂ denotes the second user information, and α₂, β₂, and γ₂ are predetermined positive real-valued coefficients. P₂ denotes the both-foot weight information, S denotes the foot size information, and B denotes the foot width information.

The user's body weight may change, and although foot size and/or foot width may not change significantly, they may be recognized differently depending on the usage environment of the walking mat 110. Therefore, the processor 430 may normalize the standing-related data and generate it as a single value representing the second user information.

In addition, the processor 430 may compare the second user information with predetermined normal standing information to determine whether the sensing information corresponds to an authorized user. For example, the processor 430 may determine that the sensing information is from an unauthorized user if the difference between the second user information and the normal standing information exceeds a preset error range. The normal standing information may be pre-stored in the memory 420. The normal standing information may be automatically set during the initial setup of the walking mat 110 based on the user's both-foot weight, foot size, and/or foot width. In this case, the initial values of α₂, β₂, and γ₂ may be preset default values.

The initially set values of α₂, β₂, and γ₂ may subsequently be adjusted based on the second user information generated over time. That is, if the difference between the second user information and the normal standing information exceeds the preset error range, the processor 430 may output an unauthorized user notification. The processor 430 may transmit the generated unauthorized user notification to the walking mat 110 or control the display 440 to output the notification. When the processor 430 transmits the unauthorized user notification to the walking mat 110 or outputs it via the display 440, and the authorized user notification is received within a predetermined time, the processor may update the average value of the normal standing information using the second user information that exceeds the error range.

Furthermore, the processor 430 may adjust the coefficients α₂, β₂, and γ₂ so that the second user information becomes equal to the updated normal standing information. For example, the updated normal standing information may be adjusted by half the difference from the second user information. Accordingly, the processor 430 may change α₂, β₂, and γ₂ so that the second user information matches the updated normal standing information. Multiple combinations of α₂, β₂, and γ₂ may result in the second user information becoming equal to the updated normal standing information. In such cases, the processor 430 may select the combination in which the value of α₂ is the largest.

If the authorized user notification is not received within a predetermined time after the unauthorized user notification is transmitted, the processor 430 may recognize that the user is in a critical security situation and transmit an emergency message to a pre-registered contact.

As described above, the present invention not only assists with walking for the elderly through a walking mat, but also enables automatic recognition of accidents involving elderly individuals living alone and allows help to be requested automatically to a guardian.

The walking condition judging method according to the present invention may be implemented in the form of computer-readable code stored on a computer-readable recording medium such as the memory 120. The computer-readable recording medium includes all types of storage media storing data that can be interpreted by a computer system. Examples include ROM (Read Only Memory), RAM (Random Access Memory), magnetic tape, magnetic disk, flash memory, optical data storage devices, and the like. In addition, the computer-readable recording medium may store and execute the code in a distributed manner across computer systems connected via a computer communication network. Accordingly, the processor 130 may execute program instructions (code) stored in the memory 120 to perform the above-described walking condition judging operations.

While the present invention has been described in detail with reference to preferred embodiments, the invention is not limited to the disclosed embodiments.

Various modifications and alterations may be made by those skilled in the art without departing from the spirit and scope of the invention.

1. A walking condition judging apparatus comprising:
a modem configured to receive sensing information from a connected walking mat and output the sensing information;
a memory in which at least one of predetermined normal walking information and predetermined normal standing information is stored; and
a processor,
wherein the memory stores program instructions executable by the processor to:
update the normal walking information or the normal standing information when the sensing information is received; and
determine whether an emergency situation has occurred when the sensing information is not received,
wherein the walking mat generates the sensing information through a pressure sensor included in the walking mat and transmits the sensing information.

2. The walking condition judging apparatus of claim 1, wherein the memory stores program instructions to:
when the sensing information is not received, compare an unrecognized accumulated time with a predetermined threshold time and determine whether the emergency situation has occurred.

3. The walking condition judging apparatus of claim 2, wherein the memory stores program instructions to:
generate a confirmation signal when the accumulated time exceeds the predetermined threshold time; and
transmit the confirmation signal to the walking mat,
wherein the walking mat outputs a user confirmation request when the confirmation signal is received.

4. The walking condition judging apparatus of claim 3, wherein the memory stores program instructions to:
determine that the situation is not an emergency when a response message is received,
wherein the walking mat transmits the response message to the modem when a user operation is input after the user confirmation request is output.

5. The walking condition judging apparatus of claim 3, wherein the memory stores program instructions to:
transmit an emergency message to a pre-registered contact when the response message is not received within a predetermined time after the confirmation signal is transmitted.

6. The walking condition judging apparatus of claim 1, wherein the memory stores program instructions to:
generate at least one of single-foot weight information, stride information, and walking speed information according to a predetermined method using the sensing information when the sensing information is received and one foot is recognized; and
determine whether the information corresponds to an authorized user based on the generated information.

7. The walking condition judging apparatus of claim 6, wherein the memory stores program instructions to:
generate first user information using the following Equation 1; and
determine whether the user is an authorized user by comparing the first user information with the normal walking information.
$${U}_{1} = {α}_{1} {P}_{1} + {β}_{1} W + {γ}_{1} V \qquad [ Equation 1 ]$$
where:
U₁ is the first user information,
α₁, β₁, and γ₁ are predetermined positive real coefficients,
P₁ is the single-foot weight information,
W is the stride information,
V is the walking speed information.

8. The walking condition judging apparatus of claim 7, wherein the memory stores program instructions to:
output an unauthorized user notification when a difference between the first user information and the normal walking information exceeds a predetermined error range; and
when an authorized user notification is input based on a user operation in response to the unauthorized user notification, update an average value of the normal walking information using the first user information, and adjust α₁, β₁, and γ₁ such that the first user information becomes equal to the updated normal walking information.

9. The walking condition judging apparatus of claim 8, wherein the memory stores program instructions to:
transmit an emergency message to a pre-registered contact when the authorized user notification is not input.

10. The walking condition judging apparatus of claim 1, wherein the memory stores program instructions to:
generate at least one of both-foot weight information, foot size information, and foot width information using the sensing information according to a predetermined method when both feet are recognized and one foot is not recognized within a predetermined time; and
determine whether the information corresponds to an authorized user based on the generated information.

11. The walking condition judging apparatus of claim 10, wherein the memory stores program instructions to:
generate second user information using the following Equation 2; and
determine whether the user is an authorized user by comparing the second user information with the normal standing information.
$${U}_{2} = {α}_{2} {P}_{2} + {β}_{2} S + {γ}_{2} B \qquad [ Equation 2 ]$$
where:
U₂ is the second user information,
α₂, β₂, and γ₂ are predetermined positive real coefficients,
P₂ is the both-foot weight information,
S is the foot size information,
B is the foot width information.

12. The walking condition judging apparatus of claim 11, wherein the memory stores program instructions to:
output an unauthorized user notification when a difference between the second user information and the normal standing information exceeds a predetermined error range; and
when an authorized user notification is input based on a user operation in response to the unauthorized user notification, update an average value of the normal standing information using the second user information, and adjust α₂, β₂, and γ₂ such that the second user information becomes equal to the updated normal standing information.

13. The walking condition judging apparatus of claim 12, wherein the memory stores program instructions to:
transmit an emergency message to a pre-registered contact when the authorized user notification is not input.
